(19) United States
(12) Patent Application Publication
(10) Pub. No.: US 2011/0038939 A1
(43) Pub. Date: Feb. 17, 2011

(54) THERAPEUTIC STABLE NANOPARTICLES

(75) Inventors: Yuri Lvov, Ruston, LA (US); Vladimir Torchilin, Charlestown, MA (US); Anshul Agarwal, Shreveport, LA (US)

Correspondence Address: WILMERHALE/BOSTON, 60 STATE STREET, BOSTON, MA, 02109, US

(73) Assignees: NORTHEASTERN UNIVERSITY, Boston, MA (US); Louisiana Tech University Research Foundation, a division of Louisiana Tech University Foundation, Ruston, LA (US)

(21) Appl. No.: 12/669,395

(22) PCT Filed: Jul. 16, 2008
(86) PCT No.: PCT/US08/70164

§ 371 Date: Oct. 25, 2010

Related U.S. Application Data
(60) Provisional application No. 60/959,728, filed on Jul. 16, 2007.

(51) Int. Cl. A61K 9/14; A61K 39/395; A61P 35/00; B82Y 5/00

(52) U.S. Cl. 424/490; 424/130.1; 424/172.1; 977/773

(57) ABSTRACT

Stable colloid nanoparticles comprising poorly soluble drugs are disclosed, as well as methods of making and methods of using such nanoparticles, e.g., as therapeutics and diagnostics.

### CROSS-REFERENCE TO RELATED APPLICATIONS

This application claims the benefit of priority to U.S. Provisional Application No. 60/959,728, filed Jul. 16, 2007, the contents of which are incorporated by reference herein in their entirety.

### FIELD OF THE INVENTION

The invention is in the field of therapeutic nanoparticles for medical screening and treatment.

### BACKGROUND OF THE INVENTION

Many potent drugs and drug candidates, especially anticancer drugs, are poorly soluble in water (e.g., tamoxifen, paclitaxel, and camptothecin). Their poor solubility results in their low bioavailability and difficulties in preparing dosage forms.

Current attempts to solve this problem are associated with loading poorly soluble drugs (usually hydrophobic molecules) into various nanosized pharmaceutical carriers such as liposomes (drugs are loaded into the hydrophobic membrane of the liposome), micelles (drugs are loaded into the hydrophobic core of the micelle), and oil-in-water emulsions. However, many general problems are associated with these approaches. For example, the nanocarriers exhibit relatively low loading efficacy of the drug into the nanocarrier (between 0.5% and 25% by weight, and often below 10% by weight); the protocols cannot be standardized, since each drug requires its own specific conditions for solubilization; scaling up the technology is difficult; controlling surface properties or surface composition of such nanosystems is difficult; and the nanocarriers have insufficient storage stability and demonstrate instability in the body.

### SUMMARY OF THE INVENTION

The invention is based, at least in part, on the discovery of a universal platform for making stable nanocolloids containing high concentration of poorly water soluble drugs. This discovery was exploited to develop the invention, which, in one aspect, features a nanoparticle comprising a compound or drug, and one or bilayers composed of; a first defined solid polymeric layer comprising a first polymer, the first layer surrounding the compound; and a second defined solid polymeric layer comprising a second polymer, the second layer surrounding the first layer, the first polymer and the second polymer having opposite charges, and the nanoparticle having a diameter of between about 100 nm and about 500 nm. In other embodiments, each layer can be composed of more than one polymer having similar isoelectric points.

In certain embodiments, the nanoparticle has a diameter of between about 100 nm and about 450 nm, between about 100 nm and about 400 nm, between about 100 nm and about 300 nm, between about 100 nm and about 250 nm, between about 100 nm and about 200 nm, between about 100 nm and about 150 nm, or about 100 nm.

In some embodiments, the compound is present in the nanoparticle between about 5% by weight and about 95% by weight, between about 20% by weight and about 90% by weight, between about 40% by weight and about 85% by weight, between about 60% by weight and about 85% by weight, between about 75% by weight to about 90% by weight, and between about 80% by weight and about 90% by weight.

In other embodiments, the first polymeric layer and the second polymeric layer have a combined thickness of between about 5 nm and about 30 nm, between about 5 nm and about 25 nm, between about 5 nm and about 20 nm, between about 5 nm and about 15 nm, and between about 5 nm and about 10 nm.

In certain embodiments, the first polymer is positively charged and the second polymer is negatively charged. In other embodiments, the first polymer is negatively charged and the second polymer is positively charged.

In some embodiments, the compound is a therapeutic compound described herein. In one embodiment, the compound is a cancer therapeutic described herein. In particular embodiments, the compound is tamoxifen or paclitaxel. In other embodiments, the compound is a low soluble anticancer drugs, camptothecin, topotecan, irinotecan, KRN 5500 (KRN), meso-tetraphenylporphine, dexamethasone, a benzodiazepine, allopurinol, acetohexamide, benzthiazide, chlorpromazine, chlordiazepoxide, haloperidol, indomethacine, lorazepam, methoxsalen, methylprednisone, nifedipine, oxazepam, oxyphenbutazone, prednisone, prednisolone, pyrimethamine, phenindione, sulfisoxazole, sulfadiazine, temazepam, sulfamerazine, ellipticin, porphine derivatives for photo-dynamic therapy, and/or trioxsalen. In some embodiments, the nanoparticle contains more than one type of compound.

In yet other embodiments, the first polymer is poly(dimethyldiallylamide ammonium chloride) (PDDA), poly(allylamine hydrochloride) (PAH), or protamine sulfate (PS). In certain embodiments, the first polymer is poly(allylamine), poly(dimethyldiallyammonim chloride) polylysine, poly(ethylenimine), poly(allylamine), dextran amine, polyarginine, chitosan, gelatine A, or protamine sulfate. In some embodiments, the second polymer is sodium poly(styrene sulphonate) (PSS) or human serum albumin (HSA). In particular embodiments, the second polymer is polyglutamic or alginic acids, poly(acrylic acid), poly(aspartic acid), poly(glutaric acid), dextran sulfate, carboxymethyl cellulose, hyaluronic acid, sodium alginate, gelatine B, chondroitin sulfate, and/or heparin.

In certain embodiments, the first polymer is a biocompatible and/or biodegradable polymer. In other embodiments, the second polymer is a biocompatible and/or biodegradable polymer. In other embodiments, both the first and the second polymer are biocompatible and/or biodegradable.

In yet other embodiments, the nanoparticle further comprises a third polymeric layer surrounding the second polymeric layer. In particular embodiments, the third polymeric layer comprises a third polymer having an opposite charge from the second polymer. In some embodiments, the third polymeric layer comprises PDDA. In certain embodiments, the first polymer and the third polymer are the same.

In other embodiments, the compound is poorly soluble in water. In particular embodiments, the compound has a solubility in aqueous medium of less than about 10 mg/mL, of less than about 5 mg/mL, of less than about 2.5 mg/mL, of less than about 1 mg/mL, or of less than about 0.5 mg/mL.

In some embodiments, outermost polymeric layer is modified with a targeting agent. In certain embodiments, the targeting agent is an antibody. In particular embodiments, the antibody is an antibody against IL2 receptor a, complement system protein C5, CD11a, CD20, TNF-alpha, T cell CD3 receptor, T cell VLA4 receptor, F protein of RSV, epidermal growth factor receptor, vascular endothelial growth factor, glycoprotein IIb/IIIa, CD52, or epidermal growth factor receptor. In other embodiments, the antibody is a monoclonal 2C5 antibody.

In some embodiments, the nanoparticle does not contain a detergent, surfactant, or oil.

In other embodiments, the compound is released from the nanoparticle at a rate of about 9%, about 7%, about 6%-4%, and about 3% with coatings of one, two, three, and four bilayers of polymers, respectively, in about two hours.

In another aspect, the invention features a nanoparticle comprising a compound; and a polymeric coating comprising alternating polymeric layers of oppositely charged polymers; the nanoparticle having a diameter of about 100 nm to about 500 nm. In certain embodiments, the nanoparticle comprises two, three, four, five, or six polymeric layers of oppositely charged polymers.

In certain embodiments, the nanoparticle has a diameter of between about 100 nm and about 450 nm, between about 100 nm and about 400 nm, between about 100 nm and about 300 nm, between about 100 nm and about 250 nm, between about 100 nm and about 200 nm, between about 100 nm and about 150 nm, or about 100 nm.

In some embodiments, the polymers are polymers described herein. In particular embodiments, the nanoparticle comprises a first polymeric layer comprising poly(dimethyldiallylamide ammonium chloride) (PDDA), poly(allylamine hydrochloride) (PAH), or protamine sulfate (PS). In other embodiments, the nanoparticle comprises a second polymeric layer comprising sodium poly(styrene sulphonate) (PSS) or human serum albumin (HSA). In yet other embodiments, the nanoparticle comprises a third polymeric layer comprising poly(dimethyldiallylamide ammonium chloride) (PDDA), poly(allylamine hydrochloride) (PAH), or protamine sulfate (PS). In still other embodiments, the nanoparticle comprises a fourth polymeric layer comprising sodium poly(styrene sulphonate) (PSS) or human serum albumin (HSA). And in still other embodiments, the nanoparticle comprises a fifth polymeric layer comprising poly(dimethyldiallylamide ammonium chloride) (PDDA), poly(allylamine hydrochloride) (PAH), or protamine sulfate (PS). In yet another embodiment, the nanoparticle comprises a sixth polymeric layer comprising sodium poly(styrene sulphonate) (PSS) or human serum albumin (HSA).

In other embodiments, the compound is poorly soluble in water. In particular embodiments, the compound has a solubility in aqueous medium of less than about 10 mg/mL, less than about 5 mg/mL, less than about 2.5 mg/mL, less than about 1 mg/mL, or less than about 0.5 mg/mL.

In certain embodiments, the compound is a therapeutic compound described herein. In some embodiments, the compound is tamoxifen or paclitaxel, and the compound is present between about 5% by weight and about 95% by weight, between about 20% by weight and about 90% by weight, between about 40% by weight and about 85% by weight, between about 60% by weight and about 85% by weight, between about 75% by weight to about 90% by weight, and between about 80% by weight and about 90% by weight. In some embodiments, the nanoparticle is a nanoparticle described herein.

In another aspect, the invention features a method of making a stable nanoparticle, the method comprising subjecting a water-insoluble compound to ultrasonication; and adding a first polymer to the compound in the presence of ultrasonication, the polymer added at a concentration sufficient to form a stable first polymeric layer around the compound.

In some embodiments, after ultrasonication, the water-insoluble compound has a negative charge in the absence of the polymer. In other embodiments, the polymer added to the compound has a positive charge.

In particular embodiments, the ultrasonication is performed at about 20° C. to about 30° C. In certain embodiments, the ultrasonication is performed at between about 10° C. and about 40° C., between about 15° C. and about 35° C., or between about 10° C. and about 25° C.

In certain embodiments, the nanoparticle has a diameter of between about 100 nm and about 450 nm, between about 100 nm and about 400 nm, between about 100 nm and about 300 nm, between about 100 nm and about 250 nm, between about 100 nm and about 200 nm, between about 100 nm and about 150 nm, or about 100 nm.

In other embodiments, the compound is poorly soluble in water. In particular embodiments, the compound has a solubility in aqueous medium of less than about 10 mg/mL, of less than about 5 mg/mL, of less than about 2.5 mg/mL, of less than about 1 mg/mL, or of less than about 0.5 mg/mL.

In certain embodiments, the compound is a therapeutic compound described herein. In some embodiments, the compound is tamoxifen or paclitaxel, and the compound is present between about 5% by weight and about 95% by weight, between about 20% by weight and about 90% by weight, between about 40% by weight and about 85% by weight, between about 60% by weight and about 85% by weight, between about 75% by weight to about 90% by weight, and between about 80% by weight and about 90% by weight. In some embodiments, the nanoparticle is a nanoparticle described herein.

In other embodiments, the first polymer is poly(dimethyldiallylamide ammonium chloride) (PDDA), poly(allylamine hydrochloride) (PAH), or protamine sulfate (PS). In particular embodiments, the method further comprising adding a second polymer to the nanoparticle after the first polymeric layer is formed. In some embodiments, the second polymer is sodium poly(styrene sulphonate) (PSS) or human serum albumin (HSA).

In yet another aspect, the invention features a method of treating a subject having a tumor, the method comprising administering to the subject a nanoparticle in an amount sufficient to reduce tumor size or number of tumor cells, wherein the nanoparticle comprises a compound; a first defined solid polymeric layer comprising a first polymer, the first layer surrounding the compound; and a second defined solid polymeric layer comprising a second polymer, the second layer surrounding the first layer, the first polymer and the second polymer having opposite charges, and the nanoparticle having a diameter of about 100 nm to about 500 nm.

In certain embodiments, the nanoparticle has a diameter of between about 100 nm and about 450 nm, between about 100 nm and about 400 nm, between about 100 nm and about 300 nm, between about 100 nm and about 250 nm, between about 100 nm and about 200 nm, between about 100 nm and about 150 nm, or about 100 nm.

In certain embodiments, the compound is a therapeutic compound described herein. In some embodiments, the compound is tamoxifen or paclitaxel, and the compound is present between about 5% by weight and about 95% by weight, between about 20% by weight and about 90% by weight, between about 40% by weight and about 85% by weight, between about 60% by weight and about 85% by weight, between about 75% by weight to about 90% by weight, and between about 80% by weight and about 90% by weight. In some embodiments, the nanoparticle is a nanoparticle described herein.

In some embodiments, the subject is a vertebrate. In certain embodiments, the subject is a mammal. In particular embodiments, the subject is a human.

The following figures are presented for the purpose of illustration only, and are not intended to be limiting.

### BRIEF DESCRIPTION OF THE DRAWINGS

FIG. 1A is a diagrammatic representation of a method for making a nanoparticle of the invention.

FIG. 1B is a diagrammatic representation of a method of conjugation of an antibody to a nanoparticle of the invention.

FIG. 2 is a graphic representation of the particle size of nanoparticles containing tamoxifen or paclitaxel particle size following various durations of sonication.

FIG. 3 is a graphic representation of the zeta potential obtained from tamoxifen particles (5 mg/mL) after normal water bath sonication or pulse power sonication.

FIG. 4 is a graphic representation of zeta potentials obtained from serial additions of PDDA or PSS onto tamoxifen (2 mg/mL) nanoparticles.

FIG. 5 is a graphic representation of zeta potentials obtained from the addition of PAH and PDDA onto paclitaxel (2.5 mg/mL)-containing nanoparticles.

FIG. 6 is a graphic representation of zeta potentials obtained from serial additions of PAH and PSS onto paclitaxel (4 mg/mL)-containing nanoparticles.

FIG. 7A is a representation of a scanning electron microscopy (SEM) image of tamoxifen-containing nanoparticles with 2 mg/mL PAH at low magnification.

FIGS. 7B and 7C are representations of SEM images of two tamoxifen-containing nanoparticles at higher magnification.

FIG. 8 is a representation of an SEM image of tamoxifen coated with polyanion PSS.

FIG. 9A is a representation of an SEM image of paclitaxel (2 mg/mL) sonicated for 10 min at 18 watts on ice without any polyelectrolyte.

FIG. 9B is a representation of an SEM image of paclitaxel (2 mg/mL) sonicated for 10 min at 18 watts surrounded by liquid nitrogen without any polyelectrolyte.

FIG. 9C is a representation of an SEM image of paclitaxel (2 mg/mL) particles obtained after two bilayer deposition (PAH-PSS)₂ surrounded by liquid nitrogen.

FIG. 9D is a representation of an SEM image of paclitaxel (2 mg/mL) particles obtained after two bilayer deposition (PAH-PSS)₂ surrounded by liquid nitrogen.

FIG. 10 is a representation of a confocal fluorescent image of an aqueous dispersion of tamoxifen-containing nanoparticles coated with FITC-labeled PAH.

FIG. 11 is a representation of a confocal fluorescent image of a tamoxifen-containing nanoparticle having a shell composition of PAH-PSS-PAH, with the third PAH layer labeled with FITC.

FIG. 12 is a graphic representation of the release of tamoxifen over time from tamoxifen alone without sonication, tamoxifen alone with sonication, tamoxifen-containing nanoparticles having a single PDDA layer, or tamoxifen-containing nanoparticles with (PDDA-PSS)₃ bilayers.

FIG. 13 is a graphic representation of the release of paclitaxel over time from naked paclitaxel with sonication, paclitaxel-containing nanoparticles having one PDDA layer, or paclitaxel-containing nanoparticles having (PDDA-PSS)₃ bilayers.

FIG. 14 is a graphic representation of an ELISA assay for different concentrations of paclitaxel-containing nanoparticles, paclitaxel-containing nanoparticles modified with mAb 2C5, or with increasing concentrations of native mAb 2C5.

FIG. 15 is a graphic representation of zeta potentials of meso-tetraphenylporphyrin-containing nanoparticles coated with FITC-PAH.

FIG. 16 is a graphic representation of particle size of camptothecin-containing nanoparticles coated with PAH, PDDA, poly L-lysine, PSS, or uncoated.

FIG. 17 is a graphic representation of zeta potentials of paclitaxel-containing nanoparticles coated with PS, (PS-HSA)₁, (PS-HSA)₁PS, or (PS-HSA)₂.

FIG. 18 is a graphic representation of paclitaxel release over time from naked paclitaxel with sonication, paclitaxel-containing nanoparticles with one layer of PDDA, paclitaxel-containing nanoparticles having (PS-HSA)₂ layers, or paclitaxel-containing nanoparticles having (PDDA-PSS)₃ layers.

FIG. 19 is a graphic representation of paclitaxel release over time from paclitaxel-containing nanoparticles coated with (PS-HSA)₃ layers.

### DETAILED DESCRIPTION OF THE INVENTION

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein, including GenBank database sequences, are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DEFINITIONS

The term “protein” is used interchangeably herein with the terms “peptide” and “polypeptide”.

As used herein, a “subject” is a mammal, e.g., a human, mouse, rat, guinea pig, dog, cat, horse, cow, pig, or non-human primate, such as a monkey, chimpanzee, baboon or rhesus.

As used herein, the term “biodegradable” refers to a substance that is decomposed (e.g., chemically or enzymatically) or broken down in component molecules by natural biological processes (e.g., in vertebrate animals such as humans).

As used herein, the term “biocompatible” refers to a substance that has no unintended toxic or injurious effects on biological functions in a target organism.

The term “targeting agent” refers to a ligand or molecule capable of specifically or selectively (i.e., non-randomly) binding or hybridizing to, or otherwise interacting with, a desired target molecule. Examples of targeting agents include, but are not limited to, nucleic acid molecules (e.g., RNA and DNA, including ligand-binding RNA molecules such as aptamers, antisense, or ribozymes), polypeptides (e.g., antigen binding proteins, receptor ligands, signal peptides, and hydrophobic membrane spanning domains), antibodies (and portions thereof), organic molecules (e.g., biotin, carbohydrates, and glycoproteins), and inorganic molecules (e.g., vitamins). A nanoparticle described herein can have affixed thereto one or more of a variety of such targeting agents.

As used herein, the term “nanoparticle” refers to a particle having a diameter in the range of about 50 nm to about 1000 nm. Nanoparticles include particles capable of containing a therapeutic or diagnostic agent that can be released within a subject. The terms “nanoparticle” and “nanocolloids” are used interchangeably herein.

As used herein, “about” means a numeric value having a range of ±10% around the cited value.

As used herein, “treat,” “treating” or “treatment” refers to administering a therapy in an amount, manner (e.g., schedule of administration), and/or mode (e.g., route of administration), effective to improve a disorder (e.g., a disorder described herein) or a symptom thereof, or to prevent or slow the progression of a disorder (e.g., a disorder described herein) or a symptom thereof. This can be evidenced by, e.g., an improvement in a parameter associated with a disorder or a symptom thereof, e.g., to a statistically significant degree or to a degree detectable to one skilled in the art. An effective amount, manner, or mode can vary depending on the subject and may be tailored to the subject. By preventing or slowing progression of a disorder or a symptom thereof, a treatment can prevent or slow deterioration resulting from a disorder or a symptom thereof in an affected or diagnosed subject.

As used herein, a “solid” layer refers to a defined firm border between a compound within a nanoparticle and the environment external to the compound. For example, nanoparticles described herein can have one or more solid polymeric layers that reduce or restrict the access of external molecules to the compound at the core of the nanoparticle.

The term “polymer,” as used herein, refers to a molecule composed of repeated subunits. Such molecules include, but are not limited to, polypeptides, polynucleotides, polysaccharides or polyalkylene glycols. Polymers can also be biodegradable and/or biocompatible.

The terms “polypeptide,” “peptide,” and “protein” are used interchangeably herein and refer to a polymer of amino acid residues. The terms apply to naturally occurring amino acid polymers as well as amino acid polymers in which one or more amino acid residues are non-natural amino acids. Additionally, such polypeptides, peptides, and proteins include amino acid chains of any length, including full length proteins, wherein the amino acid residues are linked by covalent peptide bonds.

As used herein, “stable” means that, for a period of at least six months after the nanoparticles are made, a majority of the nanoparticles remain intact at RT in a non-suspended form or as a dry pellet.

As used herein, a compound that is “poorly soluble,” when referring to a compound, means a compound that has a solubility in aqueous medium of less than about 10 mg/mL, such as less than about 1 mg/mL.

The term “drug,” as used herein, refers to any substance used in the prevention, diagnosis, alleviation, treatment, or cure of a disease or condition.

As used herein, “zeta potential” means the electric potential across an ion layer, e.g., a charged polymeric layer, around a charged colloidal nanoparticle.

The term “surrounding” is used herein to mean enclosing, enveloping, encompassing, or extending around at least a portion of the drug or compound or interior layer.

The methods described herein use, in part, a layer-by-layer (LBL) coating technology to make stable colloids of poorly soluble drugs. For this purpose, aqueous suspensions of poorly soluble drugs with a particle size of the order of microns are subjected physical treatment, such as ultrasonic treatment or ball milling (crushing), to decrease the size of individual particles to the nanolevel (e.g., between about 25 nm and about 1000 nm, between about 100 nm and about 500 nm, or between about 100 nm and about 200 nm), which are then stabilized in solution by the formation of a thin polymeric layer (or layers) on their surface. This polymeric layer (or layers) prevents particle agglomeration after stopping the physical treatment, which results in the formation of stable colloidal dispersions with high drug content in each colloidal particle (e.g., more than about 50% by weight and up to about 90% by weight). The polymeric coating is formed based on a polyelectrolyte complexing process, when drug nanosuspensions formed by, for example, ultrasonication, are incubated in the presence of a water soluble, polymer (polycation or polyanion) to allow for its deposition on their surface. The first polymeric layer can then be stabilized by the addition of another, oppositely-charged polyelectrolyte, which forms a firm electrostatic complex with the first layer (i.e., a “bilayer”). This results in the appearance of a very thin, but stable, polymeric layer or shell around each nanoparticle of a compound. This shell can prevent particle agglomeration, and can be easily and reproducibly formed on the surface of any compound particle. By varying the charge density on each polymer, or the number of coating cycles, drug particles can be prepared with a different surface charge and different thickness of the polymeric coat. This, in turn, provides a way to control drug release from such particles.

The formation of alternate outermost layers of the opposite charge at every adsorption cycle is part of the procedure. An alternate assembly of linear polyanions and polycations typically provides 1-2 nm growth step for a single bilayer, and a number of bilayers, which can be built up, can vary from one to few hundreds.

### Compounds

A nanoparticle as described herein can contain many types of compounds, such as therapeutic drugs or agents. Such therapeutic agents can be, but are not limited to, steroids, analgesics, local anesthetics, antibiotic agents, chemotherapeutic agents, immunosuppressive agents, anti-inflammatory agents, antiproliferative agents, antimitotic agents, angiogenic agents, antipsychotic agents, central nervous system (CNS) agents; anticoagulants, fibrinolytic agents, growth factors, antibodies, ocular drugs, and metabolites, analogs, derivatives, fragments, and purified, isolated, recombinant and chemically synthesized versions of these species, and combinations thereof.

Representative useful therapeutic agents include, but are not limited to, tamoxifen, paclitaxel, low soluble anticancer drugs, camptothecin and its derivatives, e.g., topotecan and irinotecan, KRN 5500 (KRN), meso-tetraphenylporphine, dexamethasone, benzodiazepines, allopurinol, acetohexamide, benzthiazide, chlorpromazine, chlordiazepoxide, haloperidol, indomethacine, lorazepam, methoxsalen, methylprednisone, nifedipine, oxazepam, oxyphenbutazone, prednisone, prednisolone, pyrimethamine, phenindione, sulfisoxazole, sulfadiazine, temazepam, sulfamerazine, ellipticin, porphine derivatives for photo-dynamic therapy, and/or trioxsalen, as well as all mainstream antibiotics, including the penicillin group, fluoroquinolones, and first, second, third, and fourth generation cephalosporins. These agents are commercially available from, e.g., Merck & Co., Barr Laboratories, Avalon Pharma, and Sun Pharma, among others. Nanosized colloidal suspensions of poorly soluble drugs can increase drug solubility and bioavailability.

Other agents that are useful are imaging agents such as gadolinium.

Compounds are released from a nanoparticle of the disclosure at a rate of about 9% from a one layer nanoparticle, about 7% from a two layered (or single bilayer) nanoparticle, from about 6% to about 4% from a three layered nanoparticle, or about 3% from a four layered (or two bilayer) nanoparticle.

### Polymers

The nanoparticles described herein can be produced by encapsulating a compound described herein within one or more layers of polymers, creating a defined polymeric layer. In some instances, polycation polymers are used. Such polycation polymers include, without limitation, poly(allylamine), poly(dimethyldiallyammonim chloride) polylysine, poly(ethylenimine), poly(allylamine), and natural polycations such as dextran amine, polyarginine, chitosan, gelatine A, and/or protamine sulfate. In other instances, polyanion polymers are used, including, without limitation, poly(styrenesulfonate), polyglutamic or alginic acids, poly(acrylic acid), poly(aspartic acid), poly(glutaric acid), and natural polyelectrolytes with similar ionized groups such as dextran sulfate, carboxymethyl cellulose, hyaluronic acid, sodium alginate, gelatine B, chondroitin sulfate, and/or heparin. These polymers can be synthesized, isolated, or commercially obtained.

In certain instances, biodegradable and/or biocompatible polymers are used. These include, without limitation, substantially pure carbon lattices (e.g., graphite), dextran, polysaccharides, polypeptides, polynucleotides, acrylate gels, polyanhydride, poly(lactide-co-glycolide), polytetrafluoroethylene, polyhydroxyalkonates, cross-linked alginates, gelatin, collagen, cross-linked collagen, collagen derivatives (such as succinylated collagen or methylated collagen), cross-linked hyaluronic acid, chitosan, chitosan derivatives (such as methylpyrrolidone-chitosan), cellulose and cellulose derivatives (such as cellulose acetate or carboxymethyl cellulose), dextran derivatives (such carboxymethyl dextran), starch and derivatives of starch (such as hydroxyethyl starch), other glycosaminoglycans and their derivatives, other polyanionic polysaccharides or their derivatives, polylactic acid (PLA), polyglycolic acid (PGA), a copolymer of a polylactic acid and a polyglycolic acid (PLGA), lactides, glycolides, and other polyesters, polyglycolide homoploymers, polyoxanones and polyoxalates, copolymer of poly(bis(p-carboxyphenoxy)propane)anhydride (PCPP) and sebacic acid, poly(1-glutamic acid), poly(d-glutamic acid), polyacrylic acid, poly(d1-glutamic acid), poly(1-aspartic acid), poly(d-aspartic acid), poly(d1-aspartic acid), polyethylene glycol, copolymers of the above listed polyamino acids with polyethylene glycol, polypeptides, such as, collagen-like, silk-like, and silk-elastin-like proteins, polycaprolactone, poly(alkylene succinates), poly(hydroxy butyrate) (PHB), polybutylene diglycolate), nylon-2/nylon-6-copolyamides, polydihydropyrans, polyphosphazenes, poly(ortho ester), poly(cyano acrylates), polyvinylpyrrolidone, polyvinylalcohol, poly casein, keratin, myosin, and fibrin, silicone rubbers, or polyurethanes, and the like. Other biodegradable materials that can be used include naturally derived polymers, such as acacia, gelatin, dextrans, albumins, alginates/starch, and the like; or synthetic polymers, whether hydrophilic or hydrophobic. The materials can be synthesized, isolated, and are commercially available.

### Targeting Agents

In some instances, a nanoparticle described herein includes a targeting agent that is attached, fixed, or conjugated to, the nanoparticle via the outermost layer of the nanoparticle. In certain situations, the targeting agent specifically binds to a particular biological target. Nonlimiting examples of biological targets include tumor cells, bacteria, viruses, cell surface proteins, cell surface receptors, cell surface polysaccharides, extracellular matrix proteins, intracellular proteins and intracellular nucleic acids. The targeting agents can be, for example, various specific ligands, such as antibodies, monoclonal antibodies and their fragments, folate, mannose, galactose and other mono-, di-, and oligosaccharides, and RGD peptide.

The nanoparticles and methods described herein are not limited to any particular targeting agent, and a variety of targeting agents can be used. Examples of such targeting agents include, but are not limited to, nucleic acids (e.g., RNA and DNA), polypeptides (e.g., receptor ligands, signal peptides, avidin, Protein A, and antigen binding proteins), polysaccharides, biotin, hydrophobic groups, hydrophilic groups, drugs, and any organic molecules that bind to receptors. In some instances, a nanoparticle described herein can be conjugated to one, two, or more of a variety of targeting agents. For example, when two or more targeting agents are used, the targeting agents can be similar or dissimilar. Utilization of more than one targeting agent in a particular nanoparticle can allow the targeting of multiple biological targets or can increase the affinity for a particular target.

The targeting agents can be associated with the nanoparticles in a number of ways. For example, the targeting agents can be associated (e.g., covalently or noncovalently bound) to other subcomponents/elements of the nanoparticle with either short (e.g., direct coupling), medium (e.g., using small-molecule bifunctional linkers such as SPDP (Pierce Biotechnology, Inc., Rockford, Ill.)), or long (e.g., PEG bifunctional linkers (Nektar Therapeutics, Inc., San Carlos, Calif.)) linkages. Alternatively, such agents can be directly conjugated to the outermost polymeric layer.

In addition, polymers used to produce the nanoparticles described herein can also incorporate reactive groups (e.g., amine groups such as polylysine, dextranemine, profamine sulfate, and/or chitosan). The reactive group can allow for further attachment of various specific ligands or reporter groups (e.g., ¹²⁵I, ¹³¹I, I, Br, various chelating groups such as DTPA, which can be loaded with reporter heavy metals such as ¹¹¹In, 99m-Tc, GD, Mn, fluorescent groups such as FITC, rhodamine, Alexa, and quantum dots), and/or other moieties (e.g., ligands, antibodies, and/or portions thereof). [symbol] These moieties can also be incorporated into the polymeric shell during its formation of a nanoparticle described herein.

Antibodies as Targeting Agents

In some instances, the targeting agents are antigen binding proteins or antibodies or binding portions thereof. Antibodies can be generated to allow for the specific targeting of antigens or immunogens (e.g., tumor, tissue, or pathogen specific antigens) on various biological targets (e.g., pathogens, tumor cells, normal tissue). Such antibodies include, but are not limited to, polyclonal antibodies; monoclonal antibodies or antigen binding fragments thereof; modified antibodies such as chimeric antibodies, reshaped antibodies, humanized antibodies, or fragments thereof (e.g., Fv, Fab′, Fab, F(ab′)₂); or biosynthetic antibodies, e.g., single chain antibodies, single domain antibodies (DAB), Fvs, or single chain Fvs (scFv).

Methods of making and using polyclonal and monoclonal antibodies are well known in the art, e.g., in Harlow et al., Using Antibodies: A Laboratory Manual: Portable Protocol I. Cold Spring Harbor Laboratory (Dec. 1, 1998). Methods for making modified antibodies and antibody fragments (e.g., chimeric antibodies, reshaped antibodies, humanized antibodies, or fragments thereof, e.g., Fab′, Fab, F(ab′)₂ fragments); or biosynthetic antibodies (e.g., single chain antibodies, single domain antibodies (DABs), Fv, single chain Fv (scFv), and the like), are known in the art and can be found, e.g., in Zola, Monoclonal Antibodies: Preparation and Use of Monoclonal Antibodies and Engineered Antibody Derivatives, Springer Verlag (Dec. 15, 2000; 1st edition).

In some instances, the antibodies recognize tumor specific epitopes (e.g., TAG-72 (Kjeldsen et al., Cancer Res., 48:2214-2220 (1988); U.S. Pat. Nos. 5,892,020; 5,892,019; and 5,512,443); human carcinoma antigen (U.S. Pat. Nos. 5,693,763; 5,545,530; and 5,808,005); TP1 and TP3 antigens from osteocarcinoma cells (U.S. Pat. No. 5,855,866); Thomsen-Friedenreich (TF) antigen from adenocarcinoma cells (U.S. Pat. No. 5,110,911); “KC-4 antigen” from human prostrate adenocarcinoma (U.S. Pat. Nos. 4,708,930 and 4,743,543); a human colorectal cancer antigen (U.S. Pat. No. 4,921,789); CA125 antigen from cystadenocarcinoma (U.S. Pat. No. 4,921,790); DF3 antigen from human breast carcinoma (U.S. Pat. Nos. 4,963,484 and 5,053,489); a human breast tumor antigen (U.S. Pat. No. 4,939,240); p97 antigen of human melanoma (U.S. Pat. No. 4,918,164); carcinoma or orosomucoid-related antigen (CORA) (U.S. Pat. No. 4,914,021); a human pulmonary carcinoma antigen that reacts with human squamous cell lung carcinoma but not with human small cell lung carcinoma (U.S. Pat. No. 4,892,935); T and Tn haptens in glycoproteins of human breast carcinoma (Springer et al., Carbohydr. Res., 178:271-292 (1988)), MSA breast carcinoma glycoprotein (Tjandra et al., Br. J. Surg., 75:811-817 (1988)); MFGM breast carcinoma antigen (Ishida et al., Tumor Biol., 10: 12-22 (1989)); DU-PAN-2 pancreatic carcinoma antigen (Lan et al., Cancer Res., 45:305-310 (1985)); CA125 ovarian carcinoma antigen (Hanisch et al., Carbohydr. Res., 178:29-47 (1988)); and YH206 lung carcinoma antigen (Hinoda et al., Cancer J., 42:653-658 (1988)).

For example, to target breast cancer cells, the nanoparticles can be modified with folic acid, EGF, FGF, and antibodies (or antibody fragments) to the tumor-associated antigens MUC 1, cMet receptor and CD56 (NCAM).

Other antibodies that can be used recognize specific pathogens (e.g., Legionella peomophilia, Mycobacterium tuberculosis, Clostridium tetani, Hemophilus influenzae, Neisseria gonorrhoeae, Treponema pallidum, Bacillus anthracis, Vibrio cholerae, Borrelia burgdorferi, Cornebacterium diphtheria, Staphylococcus aureus, human papilloma virus, human immunodeficiency virus, rubella virus, and polio virus).

Antibodies or ligands that can be attached to the nanoparticles described herein include, without limitation, antibodies to IL2 receptor a, complement system protein C5, CD11a, CD20, TNF-alpha, T cell CD3 receptor, T cell VLA4 receptor, F protein of RSV, epidermal growth factor receptor, vascular endothelial growth factor, glycoprotein IIb/IIIa, CD52, and epidermal growth factor receptor.

Antibody attachment to nanoparticles can be performed through standard covalent binding to free amine groups (see, e.g., Torchilin et al. (1987) Hybridoma, 6:229-240; Torchilin, et al., (2001) Biochim. Biophys. Acta, 1511:397-411; Masuko, et al., (2005), Biomacromol., 6:800-884) in the outermost polycation layer of polylysine or amine dextran.

For example, during formation of a polycation/polyanion multilayer shell, at every stage of the assembly, about 50% of pending ionized groups reacts with a previous layer, and another about 50% is free at the outermost shell providing a surface charge indicated by a given surface potential. Therefore, the number of amine or acidic reactive groups at the outermost shell may correspond to half of the pending groups in the polymer, e.g., 3,000 pending amine groups for poly(lysine) or poly(allylamine) in the outermost layer of a 100 nm diameter nanoshell. Standard methods of protein covalent binding are known, such as covalent binding through amine groups. This methodology can be found in, e.g., Protein Architecture: Interfacing Molecular Assemblies and Immobilization, editors: Lvov et al. (2000) Chapter 2, pp. 25-54.

To activate the polymer coat of the particle, a polymer can be used for the last layer of the particle which has free amino, carboxy, SH-, epoxy-, and/or other groups that can react with ligand molecules directly or after preliminary activation with, e.g., carbodiimides, SPDP, SMCC, and/or other mono- and bifunctional reagents.

Signal Peptides as Targeting Agents

In some instances, the targeting agents include a signal peptide. These peptides can be chemically synthesized or cloned, expressed and purified using known techniques. Signal peptides can be used to target the nanoparticles described herein to a discreet region within a cell. In some situations, specific amino acid sequences are responsible for targeting the nanoparticles into cellular organelles and compartments. For example, the signal peptides can direct a nanoparticle described herein into mitochondria. In other examples, a nuclear localization signal is used.

Nucleic Acids as Targeting Agents

In other instances, the targeting agent is a nucleic acid (e.g., RNA or DNA). In some examples, the nucleic acid targeting agents are designed to hybridize by base pairing to a particular nucleic acid (e.g., chromosomal DNA, mRNA, or ribosomal RNA). In other situations, the nucleic acids bind a ligand or biological target. For example, the nucleic acid can bind reverse transcriptase, Rev or Tat proteins of HIV (Tuerk et al., Gene, 137(1):33-9 (1993)); human nerve growth factor (Binkley et al., Nuc. Acids Res., 23(16):3198-205 (1995)); or vascular endothelial growth factor (Jellinek et al., Biochem., 83(34): 10450-6 (1994)). Nucleic acids that bind ligands can be identified by known methods, such as the SELEX procedure (see, e.g., U.S. Pat. Nos. 5,475,096; 5,270,163; and 5,475,096; and WO 97/38134; WO 98/33941; and WO 99/07724). The targeting agents can also be aptamers that bind to particular sequences.

Other Targeting Agents

The targeting agents can recognize a variety of epitopes on preselected biological targets (e.g., pathogens, tumor cells, or normal cells). For example, in some instances, the targeting agent can be sialic acid to target HIV (Wies et al., Nature, 333:426 (1988)), influenza (White et al., Cell, 56:725 (1989)), Chlamydia (Infect. Immunol, 57:2378 (1989)), Neisseria meningitidis, Streptococcus suis, Salmonella, mumps, newcastle, reovirus, Sendai virus, and myxovirus; and 9-OAC sialic acid to target coronavirus, encephalomyelitis virus, and rotavirus; non-sialic acid glycoproteins to target cytomegalovirus (Virology, 176:337 (1990)) and measles virus (Virology, 172:386 (1989)); CD4 (Khatzman et al., Nature, 312:763 (1985)), vasoactive intestinal peptide (Sacerdote et al., J. of Neuroscience Research, 18:102 (1987)), and peptide T (Ruff et al., FEBS Letters, 211:17 (1987)) to target HIV; epidermal growth factor to target vaccinia (Epstein et al., Nature, 318: 663 (1985)); acetylcholine receptor to target rabies (Lentz et al., Science 215: 182 (1982)); Cd3 complement receptor to target Epstein-Barr virus (Carel et al., J. Biol. Chem., 265:12293 (1990)); .beta.-adrenergic receptor to target reovirus (Co et al., Proc. Natl. Acad. Sci. USA, 82:1494 (1985)); ICAM-1 (Marlin et al., Nature, 344:70 (1990)), N-CAM, and myelin-associated glycoprotein MAb (Shephey et al., Proc. Natl. Acad. Sci. USA, 85:7743 (1988)) to target rhinovirus; polio virus receptor to target polio virus (Mendelsohn et al., Cell, 56:855 (1989)); fibroblast growth factor receptor to target herpes virus (Kaner et al., Science, 248:1410 (1990)); oligomannose to target Escherichia coli; and ganglioside G_{M1} to target Neisseria meningitides.

In other instances, the targeting agent targets nanoparticles according to the disclosure to factors expressed by oncogenes. These can include, but are not limited to, tyrosine kinases (membrane-associated and cytoplasmic forms), such as members of the Src family; serine/threonine kinases, such as Mos; growth factor and receptors, such as platelet derived growth factor (PDDG), SMALL GTPases (G proteins), including the ras family, cyclin-dependent protein kinases (cdk), members of the myc family members, including c-myc, N-myc, and L-myc, and bcl-2 family members.

In addition, vitamins (both fat soluble and non-fat soluble vitamins) can be used as targeting agents to target biological targets (e.g., cells) that have receptors for, or otherwise take up, vitamins. For example, fat soluble vitamins (such as vitamin D and its analogs, vitamin E, Vitamin A), and water soluble vitamins (such as Vitamin C) can be used as targeting agents.

### Therapeutic Administration

The nanoparticles described herein can be used to treat (e.g., mediate the translocation of drugs into) diseased cells and tissues. In this regard, various diseases are amenable to treatment using the nanoparticles and methods described herein. An exemplary, nonlimiting list of diseases that can be treated with the subject nanoparticles includes breast cancer; prostate cancer; lung cancer; lymphomas; skin cancer; pancreatic cancer; colon cancer; melanoma; ovarian cancer; brain cancer; head and neck cancer; liver cancer; bladder cancer; non-small lung cancer; cervical carcinoma; leukemia; non-Hodgkins lymphoma, multiple sclerosis, neuroblastoma and glioblastoma; T and B cell mediated autoimmune diseases; inflammatory diseases; infections; hyperproliferative diseases; AIDS; degenerative conditions, cardiovascular diseases, transplant rejection, and the like. In some cases, the treated cancer cells are metastatic.

The route and/or mode of administration of a nanoparticle described herein can vary depending upon the desired results. Dosage regimens can be adjusted to provide the desired response, e.g., a therapeutic response.

Methods of administration include, but are not limited to, intradermal, intramuscular, intraperitoneal, intravenous, subcutaneous, intranasal, epidural, oral, sublingual, intracerebral, intravaginal, transdermal, rectal, by inhalation, or topical, particularly to the ears, nose, eyes, or skin. The mode of administration is left to the discretion of the practitioner.

In some instances, a nanoparticle described herein is administered locally. This is achieved, for example, by local infusion during surgery, topical application (e.g., in a cream or lotion), by injection, by means of a catheter, by means of a suppository or enema, or by means of an implant, said implant being of a porous, non-porous, or gelatinous material, including membranes, such as sialastic membranes, or fibers. In some situations, a nanoparticle described herein is introduced into the central nervous system, circulatory system or gastrointestinal tract by any suitable route, including intraventricular, intrathecal injection, paraspinal injection, epidural injection, enema, and by injection adjacent to the peripheral nerve. Intraventricular injection can be facilitated by an intraventricular catheter, for example, attached to a reservoir, such as an Ommaya reservoir.

This disclosure also features a device for administering a nanoparticle described herein. The device can include, e.g., one or more housings for storing pharmaceutical compositions, and can be configured to deliver unit doses of a nanoparticle described herein.

Pulmonary administration can also be employed, e.g., by use of an inhaler or nebulizer, and formulation with an aerosolizing agent, or via perfusion in a fluorocarbon or synthetic pulmonary surfactant.

In some instances, a nanoparticle described herein can be delivered in a vesicle, in particular, a liposome (see Langer, Science 249:1527-1533 (1990) and Treat et al., Liposomes in the Therapy of Infectious Disease and Cancer pp. 317-327 and pp. 353-365 (1989)).

In yet other situations, a nanoparticle described herein can be delivered in a controlled-release system or sustained-release system (see, e.g., Goodson, in Medical Applications of Controlled Release, vol. 2, pp. 115-138 (1984)). Other controlled or sustained-release systems discussed in the review by Langer, Science 249:1527-1533 (1990) can be used. In one case, a pump can be used (Langer, Science 249:1527-1533 (1990); Sefton, CRC Crit. Ref Biomed. Eng. 14:201 (1987); Buchwald et al., Surgery 88:507 (1980); and Saudek et al., N. Engl. J. Med. 321:574 (1989)).

In yet other situations, a controlled- or sustained-release system can be placed in proximity of a target of nanoparticle described herein, reducing the dose to a fraction of the systemic dose.

A nanoparticle described herein is formulated as a pharmaceutical composition that includes a suitable amount of a physiologically acceptable excipient (see, e.g., Remington's Pharmaceutical Sciences pp. 1447-1676 (Alfonso R. Gennaro, ed., 19th ed. 1995)). Such physiologically acceptable excipients can be, e.g., liquids, such as water and oils, including those of petroleum, animal, vegetable, or synthetic origin, such as peanut oil, soybean oil, mineral oil, sesame oil and the like. The physiologically acceptable excipients can be saline, gum acacia, gelatin, starch paste, talc, keratin, colloidal silica, urea and the like. In addition, auxiliary, stabilizing, thickening, lubricating, and coloring agents can be used. In one situation, the physiologically acceptable excipients are sterile when administered to an animal. The physiologically acceptable excipient should be stable under the conditions of manufacture and storage and should be preserved against the contaminating action of microorganisms. Water is a particularly useful excipient when a nanoparticle described herein is administered intravenously. Saline solutions and aqueous dextrose and glycerol solutions can also be employed as liquid excipients, particularly for injectable solutions. Suitable physiologically acceptable excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Other examples of suitable physiologically acceptable excipients are described in Remington's Pharmaceutical Sciences pp. 1447-1676 (Alfonso R. Gennaro, ed., 19th ed. 1995). The pharmaceutical compositions, if desired, can also contain minor amounts of wetting or emulsifying agents, or pH buffering agents.

Liquid carriers can be used in preparing solutions, suspensions, emulsions, syrups, and elixirs. A nanoparticle described herein can be suspended in a pharmaceutically acceptable liquid carrier such as water, an organic solvent, a mixture of both, or pharmaceutically acceptable oils or fat. The liquid carrier can contain other suitable pharmaceutical additives including solubilizers, emulsifiers, buffers, preservatives, sweeteners, flavoring agents, suspending agents, thickening agents, colors, viscosity regulators, stabilizers, or osmo-regulators. Suitable examples of liquid carriers for oral and parenteral administration include water (particular containing additives described herein, e.g., cellulose derivatives, including sodium carboxymethyl cellulose solution), alcohols (including monohydric alcohols and polyhydric alcohols, e.g., glycols) and their derivatives, and oils (e.g., fractionated coconut oil and arachis oil). For parenteral administration the carrier can also be an oily ester such as ethyl oleate and isopropyl myristate. The liquid carriers can be in sterile liquid form for administration. The liquid carrier for pressurized compositions can be halogenated hydrocarbon or other pharmaceutically acceptable propellant.

In other instances, a nanoparticle described herein is formulated for intravenous administration. Compositions for intravenous administration can comprise a sterile isotonic aqueous buffer. The compositions can also include a solubilizing agent. Compositions for intravenous administration can optionally include a local anesthetic such as lignocaine to lessen pain at the site of the injection. The ingredients can be supplied either separately or mixed together in unit dosage form, for example, as a dry lyophilized powder or water-free concentrate in a hermetically sealed container such as an ampule or sachette indicating the quantity of active agent. Where a nanoparticle described herein is administered by infusion, it can be dispensed, for example, with an infusion bottle containing sterile pharmaceutical grade water or saline. Where a nanoparticle described herein is administered by injection, an ampule of sterile water for injection or saline can be provided so that the ingredients can be mixed prior to administration.

In other circumstances, a nanoparticle described herein can be administered across the surface of the body and the inner linings of the bodily passages, including epithelial and mucosal tissues. Such administrations can be carried out using a nanoparticle described herein in lotions, creams, foams, patches, suspensions, solutions, and suppositories (e.g., rectal or vaginal). In some instances, a transdermal patch can be used that contains a nanoparticle described herein and a carrier that is inert to the nanoparticle described herein, is non-toxic to the skin, and that allows delivery of the agent for systemic absorption into the blood stream via the skin. The carrier can take any number of forms such as creams or ointments, pastes, gels, or occlusive devices. The creams or ointments can be viscous liquid or semisolid emulsions of either the oil-in-water or water-in-oil type. Pastes of absorptive powders dispersed in petroleum or hydrophilic petroleum containing a nanoparticle described herein can also be used. A variety of occlusive devices can be used to release a nanoparticle described herein into the blood stream, such as a semi-permeable membrane covering a reservoir containing the nanoparticle described herein with or without a carrier, or a matrix containing the nanoparticle described herein.

A nanoparticle described herein can be administered rectally or vaginally in the form of a conventional suppository. Suppository formulations can be made using methods known to those in the art from traditional materials, including cocoa butter, with or without the addition of waxes to alter the suppository's melting point, and glycerin. Water-soluble suppository bases, such as polyethylene glycols of various molecular weights, can also be used.

The amount of a nanoparticle described herein that is effective for treating disorder or disease is determined using standard clinical techniques known to those with skill in the art. In addition, in vitro or in vivo assays can optionally be employed to help identify optimal dosage ranges. The precise dose to be employed can also depend on the route of administration, the condition, the seriousness of the condition being treated, as well as various physical factors related to the individual being treated, and can be decided according to the judgment of a health-care practitioner. For example, the dose of a nanoparticle described herein can each range from about 0.001 mg/kg to about 250 mg/kg of body weight per day, from about 1 mg/kg to about 250 mg/kg body weight per day, from about 1 mg/kg to about 50 mg/kg body weight per day, or from about 1 mg/kg to about 20 mg/kg of body weight per day. Equivalent dosages can be administered over various time periods including, but not limited to, about every 2 hrs, about every 6 hrs, about every 8 hrs, about every 12 hrs, about every 24 hrs, about every 36 hrs, about every 48 hrs, about every 72 hrs, about every week, about every two weeks, about every three weeks, about every month, and about every two months. The number and frequency of dosages corresponding to a completed course of therapy can be determined according to the judgment of a health-care practitioner.

In some instances, a pharmaceutical composition described herein is in unit dosage form, e.g., as a tablet, capsule, powder, solution, suspension, emulsion, granule, or suppository. In such form, the pharmaceutical composition can be sub-divided into unit doses containing appropriate quantities of a nanoparticle described herein. The unit dosage form can be a packaged pharmaceutical composition, for example, packeted powders, vials, ampoules, pre-filled syringes or sachets containing liquids. The unit dosage form can be, for example, a capsule or tablet itself, or it can be the appropriate number of any such compositions in package form. Such unit dosage form can contain from about 1 mg/kg to about 250 mg/kg, and can be given in a single dose or in two or more divided doses.

### Kits

A nanoparticle described herein can be provided in a kit. In some instances, the kit includes (a) a container that contains a nanoparticle and, optionally (b) informational material. The informational material can be descriptive, instructional, marketing or other material that relates to the methods described herein and/or the use of the nanoparticles, e.g., for therapeutic benefit.

The informational material of the kits is not limited in its form. In some instances, the informational material can include information about production of the nanoparticle, molecular weight of the nanoparticle, concentration, date of expiration, batch or production site information, and so forth. In other situations, the informational material relates to methods of administering the nanoparticles, e.g., in a suitable amount, manner, or mode of administration (e.g., a dose, dosage form, or mode of administration described herein). The method can be a method of treating a subject having a disorder.

In some cases, the informational material, e.g., instructions, is provided in printed matter, e.g., a printed text, drawing, and/or photograph, e.g., a label or printed sheet. The informational material can also be provided in other formats, such as Braille, computer readable material, video recording, or audio recording. In other instances, the informational material of the kit is contact information, e.g., a physical address, email address, website, or telephone number, where a user of the kit can obtain substantive information about the nanoparticles therein and/or their use in the methods described herein. Of course, the informational material can also be provided in any combination of formats.

In addition to the nanoparticles, the kit can include other ingredients, such as a solvent or buffer, a stabilizer, or a preservative. The kit can also include other agents, e.g., a second or third agent, e.g., other therapeutic agents. The components can be provided in any form, e.g., liquid, dried or lyophilized form. The components can be substantially pure (although they can be combined together or delivered separate from one another) and/or sterile. When the components are provided in a liquid solution, the liquid solution can be an aqueous solution, such as a sterile aqueous solution. When the components are provided as a dried form, reconstitution generally is by the addition of a suitable solvent. The solvent, e.g., sterile water or buffer, can optionally be provided in the kit.

The kit can include one or more containers for the nanoparticles or other agents. In some cases, the kit contains separate containers, dividers or compartments for the nanoparticles and informational material. For example, the nanoparticles can be contained in a bottle, vial, or syringe, and the informational material can be contained in a plastic sleeve or packet. In other situations, the separate elements of the kit are contained within a single, undivided container. For example, the nanoparticles can be contained in a bottle, vial or syringe that has attached thereto the informational material in the form of a label. In some cases, the kit can include a plurality (e.g., a pack) of individual containers, each containing one or more unit dosage forms (e.g., a dosage form described herein) of the nanoparticles. The containers can include a unit dosage, e.g., a unit that includes the nanoparticles. For example, the kit can include a plurality of syringes, ampules, foil packets, blister packs, or medical devices, e.g., each containing a unit dose. The containers of the kits can be air tight, waterproof (e.g., impermeable to changes in moisture or evaporation), and/or light-tight.

The kit can optionally include a device suitable for administration of the nanoparticles, e.g., a syringe or other suitable delivery device. The device can be provided pre-loaded with nanoparticles, e.g., in a unit dose, or can be empty, but suitable for loading.

The invention is further illustrated by the following examples. The examples are provided for illustrative purposes only. They are not to be construed as limiting the scope or content of the invention in any way.

### EXAMPLES

### Example 1

### Preparation of Stable Nano-Colloids of Poorly Soluble Drugs

Stable colloids of poorly soluble drugs were prepared in order to increase their solubilization and bioavailability. To do this high power sonication poor soluble drug aqueous dispersions is used with simultaneous LbL-nanocoating. Such coating reverses and enhances a particle surface charge which prevents re-aggregation of the drug and allows getting smaller and smaller drug colloids (proportionally to the sonication time).

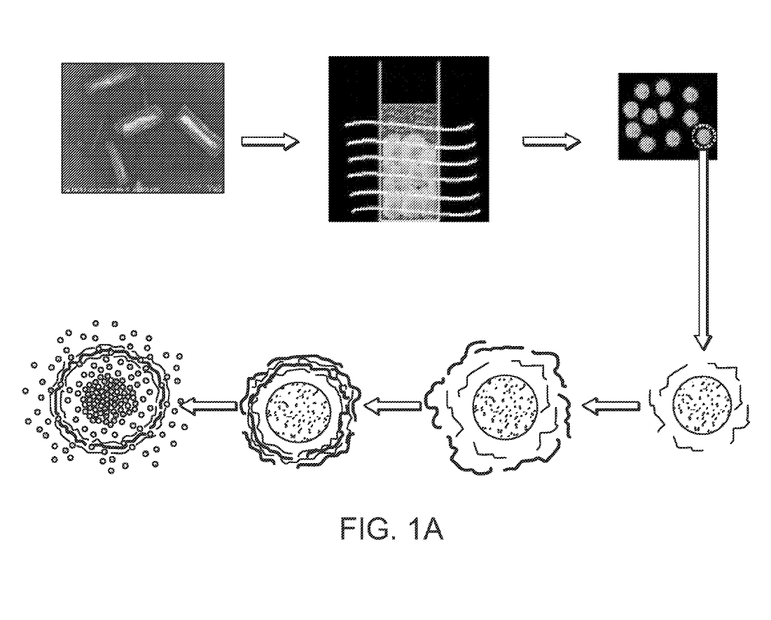

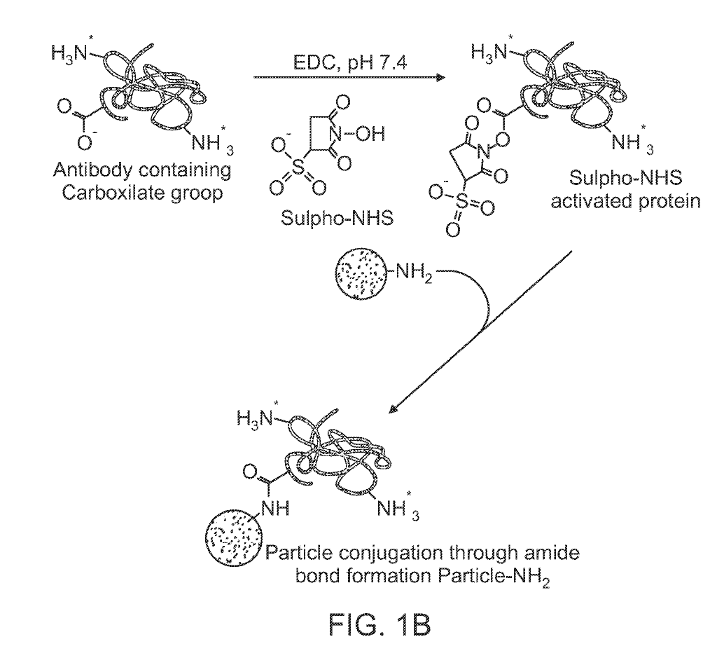

A simultaneous application of powerful sonication and adsorption of opposite charged polyelectrolytes caused a systematic decrease of insoluble drug particle size to nano-scale in the following process (depicted schematically in FIG. 1A). Sonication energy initially cleaves and cracks bulk drug, and polyelectrolytes immediately fix this sub-dividing, preventing re-aggregation of the pieces. Longer sonication times allowed smaller and smaller particles (to about 100 nm diameter) which are stable in water due to adsorbed monolayer of polyelectrolytes. Further build-up of an organized multilayer shell through layer-by-layer (LbL) architecture (alternate adsorption of polycations and polyanions) caused formation of thicker shells of about 5 nm to about 30 nm, which controlled drug release rate.

### A. Methods

Materials and Instruments

The poorly soluble and potent anti-cancer drugs tamoxifen (TMF) and paclitaxel (PCT) were used in these experiments (solubility below 1 μg/mL). All polyelectrolytes used for the LbL assembly were used at a concentration of 2 mg/mL. Poly(allylamine hydrochloride) (PAH), FITC-labeled PAH, and poly(dimethyldiallylamide ammonium chloride) (PDDA) were used as positively charged polyelectrolytes. Sodium poly(styrene sulphonate) (PSS) was used as a negatively charged polyelectrolyte. Deionized water and PBS at pH 7.4 were used as solvents. Drug crystal disintegrations were performed using an Ultra Sonicator 3000 (Misonix Inc, Farmingdale, N.Y.) at 3-18 Wt for 10-30 min. To prevent sample overheating during the sonication and to keep the temperature in the range of 20-30° C., liquid nitrogen was used to cool the sample tubes. The thickness of the polyelectrolyte multilayer was measured using a Quartz Crystal Microbalance (9 MHz QCM, USI-System, Japan). Surface potential (zeta-potential) and particle size measurements were performed using ZetaPlus Microelectrophoresis (Brookhaven Instruments). A Field Emission Scanning Electron Microscope (Hitachi, 2006) was used for particle imaging. A Laser Scanning Confocal Microscope (Leica TCS SP2 from Leica Microsystems Inc.) was also used to control shell formation and to follow colloid stability.

LbL Assembly and Properties of Nanoparticles

Initially, all drug samples were disintegrated using ultrasonication with cooling at 18 W for up to 30 mins in 1 mL volume before any polyelectrolyte was added. The size of drug particles formed was periodically measured. Prior to the addition of the first layer of polyelectrolyte, the zeta potential reading was also taken. Polycations were used to form the first surface layer, since drug nanoparticles of both drugs were found to bear an intrinsic negative charge. Drug samples were then centrifuged at 14,000 rpm for 7 min, washed, and re-suspended in either water or PBS to remove excess polyelectrolyte. Zeta potential readings were then taken. The coating process was repeated using the polyanion polymer but without ultrasonication. Zeta potential measurements were taken after each layer was added.

Images of colloidal particles formed were taken immediately and at 48 hrs following LbL assembly to analyze the stability of the colloids formed. Dry samples were prepared for SEM imaging using 5 μL-10 μL of the colloidal suspension obtained. Sample droplets on bare silicon wafers were dried by heating them at 50° C. for 1 hr or by storing them overnight at RT. Drug colloids were kept in a low volume of saturated solution to prevent drug release.

Drug Release From Colloidal Particles at Sink Conditions

To determine the release rate of different drugs from the colloidal particles prepared using LbL assembly, samples prepared using differing numbers of coating cycles were placed in 1 mL horizontal diffusion chambers made of cellulose acetate membrane. The samples were then stirred in a large volume of PBS, pH 7.2, to mimic sink conditions expected in vivo. The concentrations of the released drugs were measured by HPLC.

Attachment of Ligand Moieties to the LbL Nanocolloids of Poorly Soluble Drugs

To prepare nanocolloids with a “reactive” surface suitable for covalent attachment of various ligands, PAH containing free amino groups was used to form the outer layer on drug particles. Paclitaxel was used as the drug in this series of experiments. The monoclonal nucleosome-specific 2C5 antibody (mAb 2C5) was conjugated to LbL paclitaxel nanoparticles. This antibody recognizes a broad variety of cancer cells via cancer cell surface-bound nucleosomes (see Iakoubov et al., Oncol. Res. 9 (1997) 439-446; and Iakoubov et al., Cancer Detect. Prev. 22 (1998) 470-475). The antibody was conjugated in two steps (FIG. 1B). In the first step, the carboxylate groups on mAb 2C5 were activated using 1-ethyl-3-carbodiimide hydrochloride (EDC) and N-hydroxysulfosuccinimide (sulfo-NHS), rendering the antibody amine-reactive. In the second step, the activated antibody was added to LbL paclitaxel nanoparticles coated with polyamino-containing PAH polymer. All reactions were carried out in HBS, pH 7.4, at 4° C. with continuous stirring in the presence of argon gas. The modified particles were centrifuged at 12 rpm for 10 min and re-suspended twice using PBS to remove unconjugated antibody.

The amount of paclitaxel in the nanoparticle preparations was measured by reversed phase HPLC. A D-7000 HPLC system equipped with a diode array (Hitachi, Japan) and Spherisorb ODS2 column, 4.6 mm×250 mm (Waters, Milford, Mass., USA) was used. The particles were dissolved with the mobile phase prior to loading onto the HPLC column. The column was eluted with acetonitrile/water (65:35%, v/v) at 1.0 mL/min. A Paclitaxel peak was detected at 227 nm. Injection volume was 50 μL All samples were analyzed in triplicate.

Antibody Activity Preservation on the Surface of LbL Drug Nanoparticles

To verify the preservation of mAb 2C5 specific activity after the conjugation with LbL-paclitaxel nanoparticles, a standard ELISA was performed. Briefly, ELISA plates pretreated with 40 μg/ml polylysine solution in TBS, pH 7.4, were coated with 50 μL of 40 μg/mL nucleosomes (the water-soluble fraction of calf thymus nucleohistone, Worthington Biochemical, Lakewood, N.J.) and incubated for 1 hr at RT. The plates were then rinsed with 0.2% casein, 0.05% Tween 20 in TBS (casein/TBS), pH 7.4. To these plates, serial dilutions of mAb 2C5-containing samples were added and incubated for 1 hr at RT. The plates were extensively washed with casein/TBS and coated with horseradish peroxidase goat anti-mouse IgG conjugate (ICN Biomedical, Aurora, Ohio), diluted according to the manufacturer's recommendation. After 1 hr incubation at RT, the plates were washed with casein/TBS. Bound peroxidase was quantified by the degradation of its substrate, diaminobenzidine, supplied as a ready-for-use solution, Enhanced K-Blue TMB substrate (Neogen, Lexington, Ky.). The intensity of the color developed was analyzed using a Labsystems Multiscan MCC/340 ELISA reader at 492 nm (Labsystems and Life Sciences International, UK).

Cytotoxicity of Targeted Paclitaxel LbL Nanoparticles

The cytotoxicity of various concentrations of LbL-paclitaxel nanoparticles against MCF-7 and BT-20 cells was studied using a MTT test. A ready-for-use CellTiter 96® Aqueous One solution of MTT (Promega, Madison, Wis.) was used according to the manufacturer's protocol. Formulations with paclitaxel concentration of up to 200 ng/mL dispersed in Hank's buffer were added to cells grown in 96-well plates to about 40% confluence. After 48 hr or 72 hr of incubation at 37° C., 5% CO₂, plates were washed three times with Hank's buffer. Next, 100 μl of media and 20 μl of CellTiter 96® Aqueous One solution were added to the plates, and the plates were incubated for 1 hr at 37° C., 5% CO₂. The cell survival rate was then estimated by measuring the color intensity of the MTT degradation product at 492 nm using an ELISA plate reader. Untreated cells were considered as 100% growth.

### B. Results

LbL-Stabilized Drug Nanoparticles and Surface Zeta-Potential

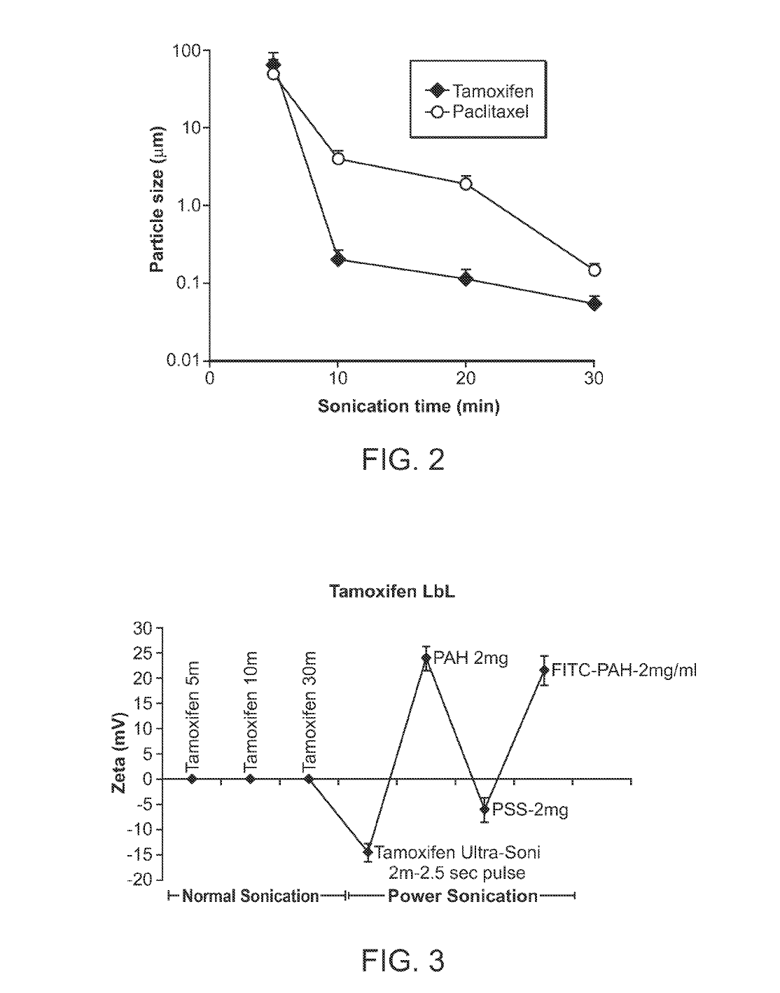

To find optimal sonication conditions, initial experiments were performed with tamoxifen crystals at a concentration of 2 mg/mL in the suspension. As shown in FIG. 2, particle size could be controlled by the duration of sonication, and decreased with increased sonication time. After 30 mins of sonication at 18 Wt, particles of about 100 nm were obtained (polycationic PDDA was added prior to the size measurement to prevent particle re-aggregation). When similar sonication conditions were applied to paclitaxel crystals, particle sizes of about 100 nm were also obtained. Increasing the sonication time further did not result in a significant decrease in drug particle size.

As depicted in FIG. 3, no surface charge for tamoxifen was observed after normal bath sonication, but a strong negative charge was obtained just after 2.5 sec pulse power sonication. Sequential addition of layers of PAH and PSS resulted in nanoparticles having positive and negative charges, respectively.

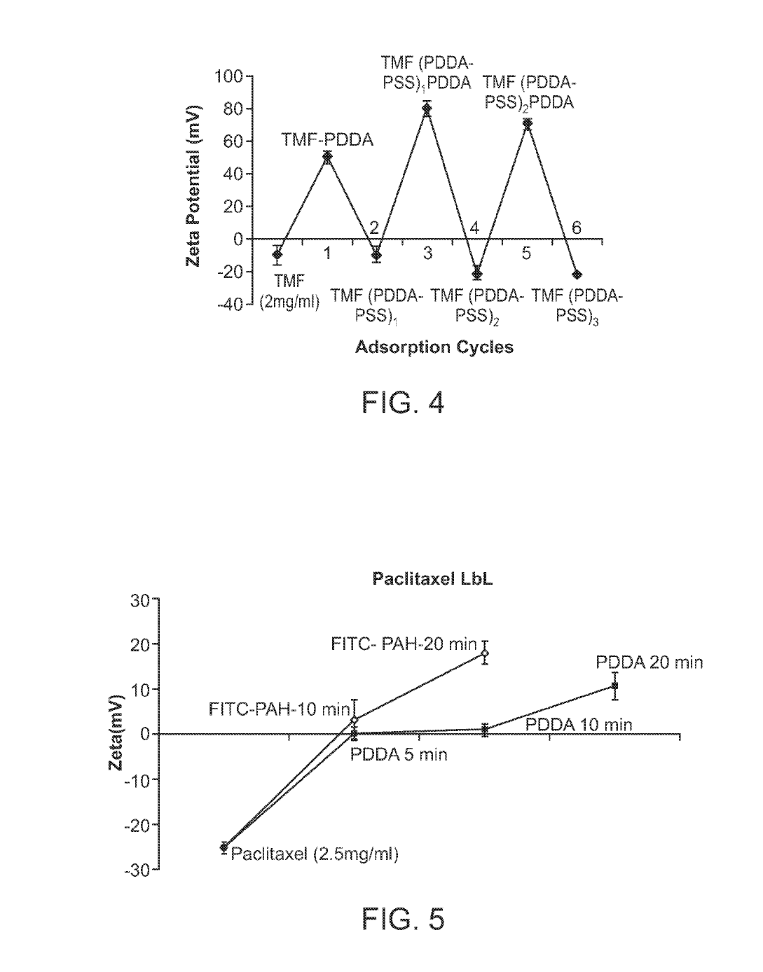

FIG. 4 depicts the values of the zeta potential measured during the process of sequential PDDA/PSS adsorption onto tamoxifen cores. After the addition of PDDA, the initially negatively charged nanoparticles were recharged to a positive potential of about +45 mV. The addition of PDDA formed a stable colloidal solution when sonication was terminated. The polyanion PSS was then added to the PDDA-coated tamoxifen nanoparticles, in the presence of sonication, to perform LbL assembly. PSS polyanion adsorption added one more monolayer to the shell, and again reversed the surface potential to a negative value (−17 mV). Next, the PDDA polycation was added again, which resulted in tamoxifen particles that were positively charged (around +80 mV). Addition of a fourth polymer layer of the polyanion PSS resulted in tamoxifen particles that were again negative. Alternating layers of PDDA and PSS were added to the tamoxifen particles until tamoxifen nanoparticles were formed that were coated with an organized multilayer shell with the composition (PDDA/PSS)₃ (FIG. 4).

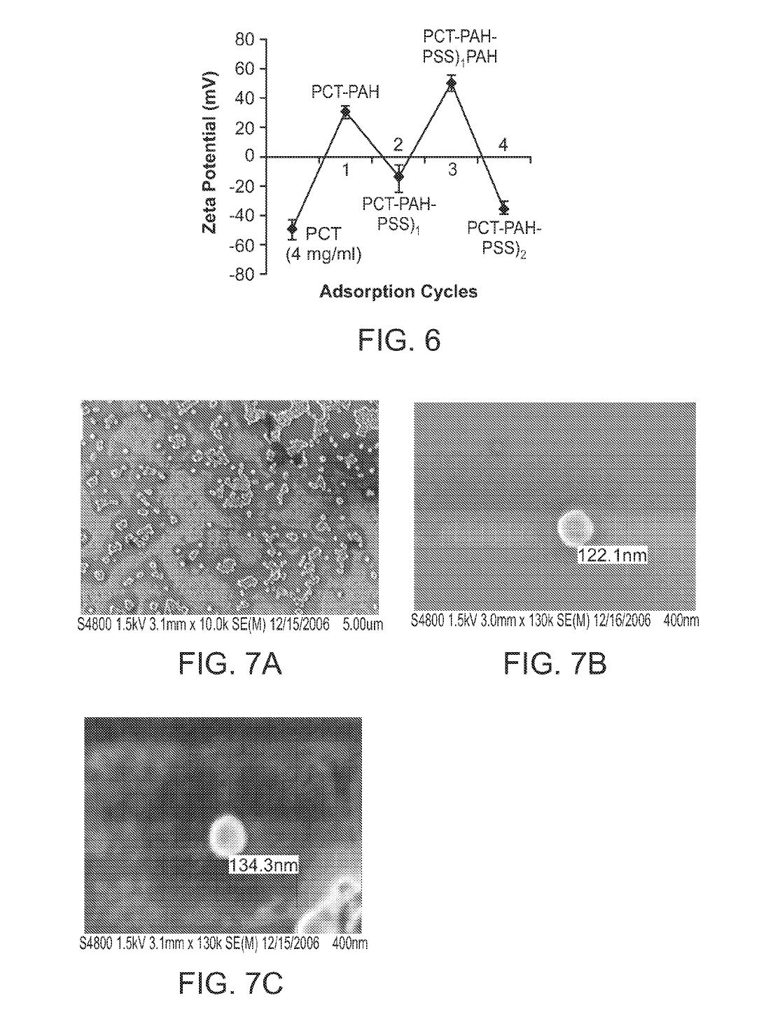

Sonicated paclitaxel particles were also initially negatively charged (FIG. 5). When paclitaxel was coated with either PAH or PDDA, the surface charge was reversed after sonication (FIG. 5). When the polyanion PSS was subsequently added to paclitaxel/PAH nanoparticles, the resulting nanoparticles had a negative zeta potential (FIG. 6). Further assembly using alternating additions of PAH and PSS under sonication resulted in nanoparticles having corresponding changes in zeta potential values, until paclitaxel nanoparticles were formed having a composition of (PAH/PSS)₂ (FIG. 6).

In separate experiments using quartz Crystal Microbalance (QCM) monitoring of the PDDA/PSS or PAH/PSS assembly on quartz resonator, a single polycation/polyanion bilayer was determined to have a thickness of 1.5 nm in dry state. As polyelectrolyte multilayer thickness doubles in water (see Decher, Science 227 (1997) 1232-1237; and Decher and Schlenoff (Eds.), Multilayer Thin Films: Sequential Assembly of Nanocomposite Materials, Wiley-VCH, Weinheim, Germany, 2003), the thickness of the (PDDA/PSS)₃ shells was estimated to be around 4.5 nm in dry state and around 9 nm in aqueous solution. (PAH/PSS)₂ shell thickness was estimated to be around 3 nm in dry state and 6 nm in aqueous solution.

Nanoparticle Imaging and Some Properties

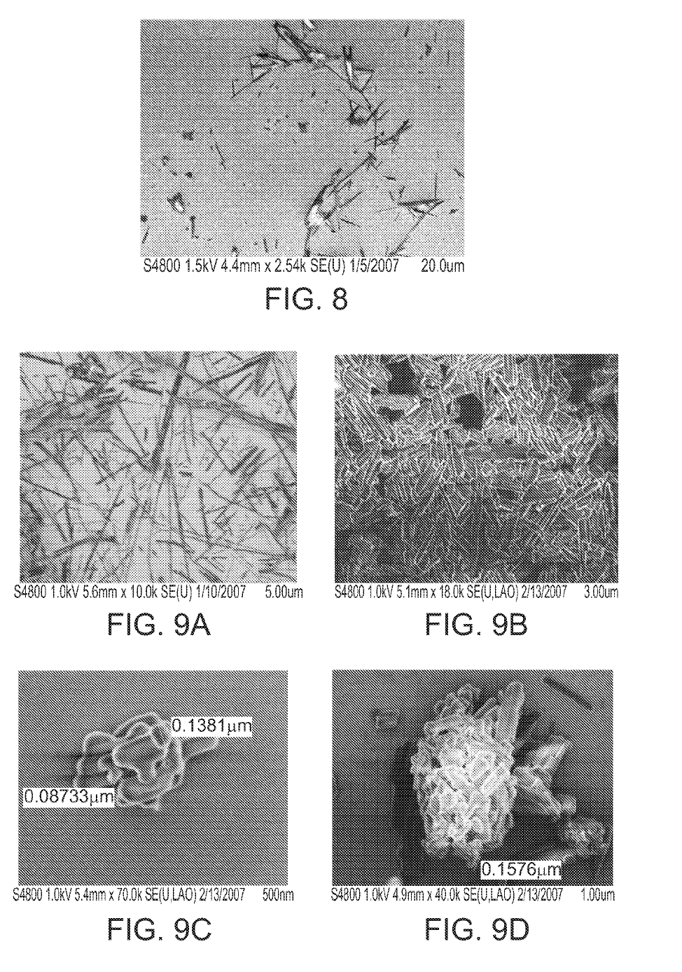

Scanning electron microscopy (SEM) and confocal fluorescence microscopy were used to confirm the sizes of the nanoparticles formed by the LbL technology described herein. After tamoxifen was sonicated for 20 mins in the presence of 2 mg/mL PAH, nanoparticles were obtained that were mainly spherical in shape and had a diameter of 120±30 nm (FIGS. 7B and 7C). The nanocolloids were stable in water, since SEM images taken after 48 hrs still showed individual non-agglomerated nanosized particles (FIGS. 7B and 7C). FIG. 8 demonstrates that adding a first layer of polyanion PSS did not result in tamoxifen size decrease even after 20 min of sonication.

For paclitaxel, nanoparticles having a (PAH-PSS)₂ shell composition were produced having particle sizes of about 87 nm and about 157 nm (FIGS. 9C and 9D). However, aggregation of some paclitaxel nanoparticles to about 1.5 μm diameter particles was observed. Reducing the initial paclitaxel concentration to 1 mg/mL resulted in nanoparticles having an elongated rod-like shape with dimensions of about 50 nm×about 120 nm, which did not aggregate.

The SEM images were obtained after drying the samples, and during the drying process the nanoparticles become partially aggregated, as depicted in FIG. 7A. To demonstrate that this aggregation was the result of SEM sample preparation and that the nanoparticles did not aggregate in aqueous suspension, images of the samples were obtained using confocal fluorescence microscopy.

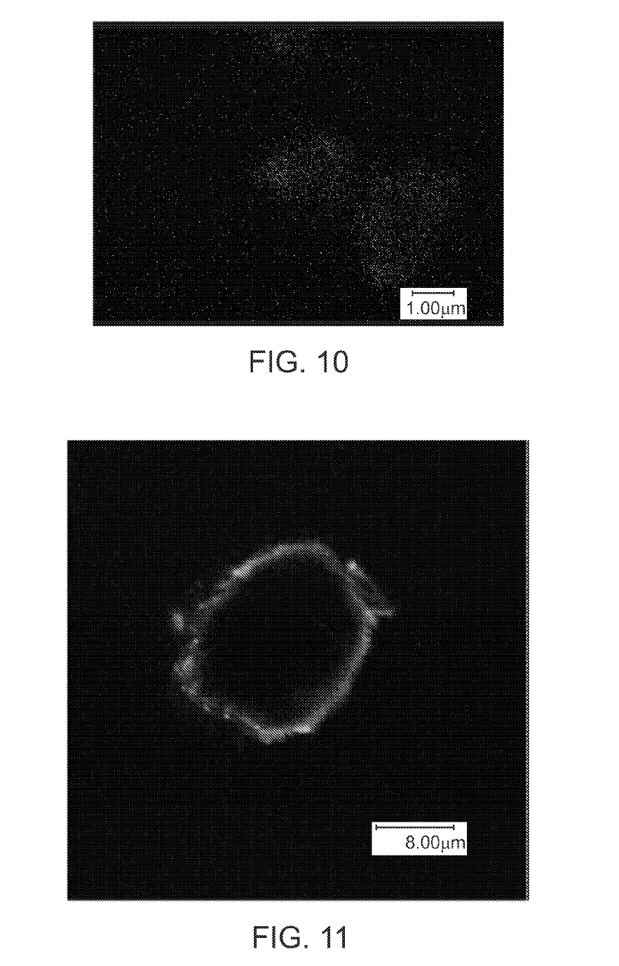

Tamoxifen nanoparticles were prepared by coating tamoxifen with a layer of FITC-labeled PAH. Fluorescence imaging of these LbL-coated tamoxifen particles in suspension did not reveal any aggregation (FIG. 10). Paclitaxel nanoparticles coated with FITC-labeled PAH also did not aggregate. Further assembly of PAH-coated tamoxifen nanoparticles through alternate sequential adsorption of PSS and PAH to build a multilayer was performed, in which the last PAH layer was labeled with FITC. FIG. 11 depicts a confocal image of a tamoxifen nanoparticle demonstrating effective LbL encapsulation within a 3-layer shell.

In other experiments, SEM and confocal images were obtained 2-7 days after sample formation, demonstrating the stability of aqueous drug nanocolloids.

Given that the thickness of a single polymeric layer was about 1.5 nm in dry state, the amount of drug in the stable nanocolloidal particles was calculated to be from about 85% by weight (for tamoxifen particles with the triple PDDA/PSS bilayer coating) to about 90% by weight (for paclitaxel particles with the double PAH/PSS layer coating). Further, colloidal suspensions of both drugs were completely stable during the two weeks of observation.

Drug Release From LbL Nanoparticles

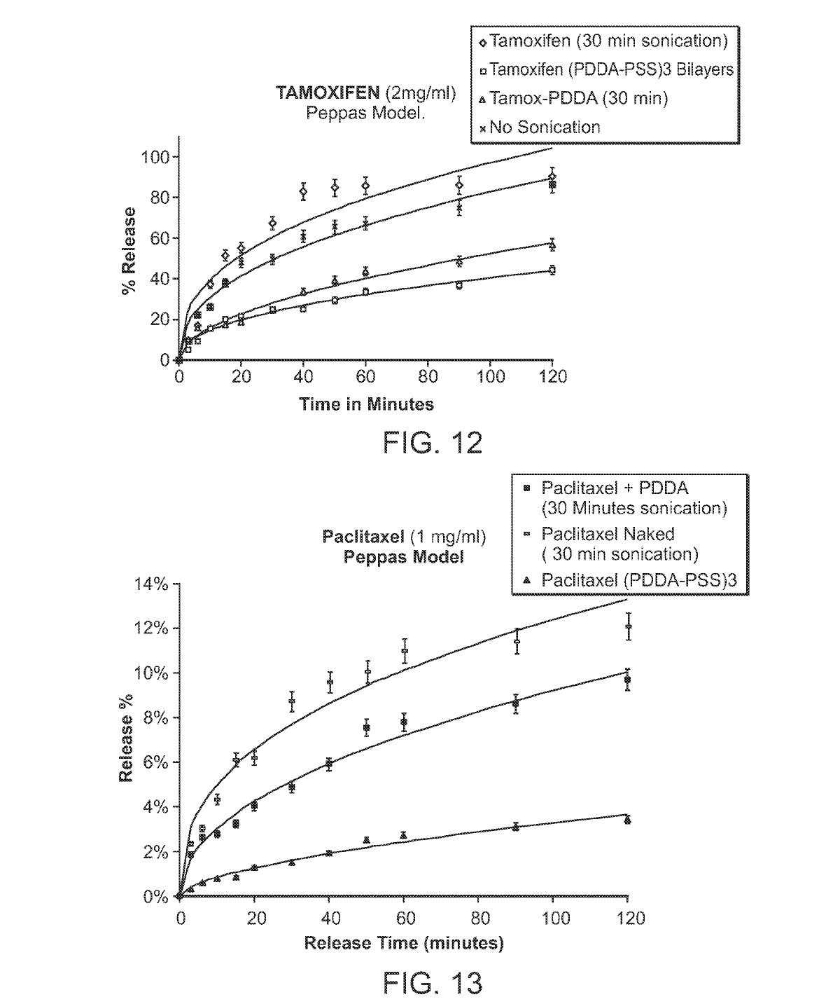

LbL technology can be used to control the drug release rate from polymer-stabilized colloidal nanoparticles by changing the thickness or composition of nanoparticles. Accordingly, the release of tamoxifen from LbL nanocolloidal particles containing 2 mg/mL tamoxifen and having a single PDDA coating or a coating composition of (PDDA/PSS)₃ was measured in standard sink conditions (PBS buffer at pH 7.4). Curves were produced from the experimental data using Peppas' model of exponential approximation (see Peppas, Pharm. Acta Helv. (1985) 60:110-112). As depicted in FIG. 12, slower release rates were observed as the number of polyelectrolyte layers in the shell increased. At sink conditions (PBS buffer at pH 7.4), non-coated tamoxifen crystals (both without and with sonication) were solubilized within about 2 hrs. PDDA- and (PDDA/PSS)₃-coated nanoparticles were estimated to solubilize at around 10 hrs. Similar results were obtained for paclitaxel. Slower release rates were obtained using LbL coatings containing different polycations and polyanions and varying the number of shells. Similar results were seen for paclitaxel nanoparticles (FIG. 13).

Surface Modification of LbL-Coated Drug Nanoparticles and Cytotoxicity Analysis

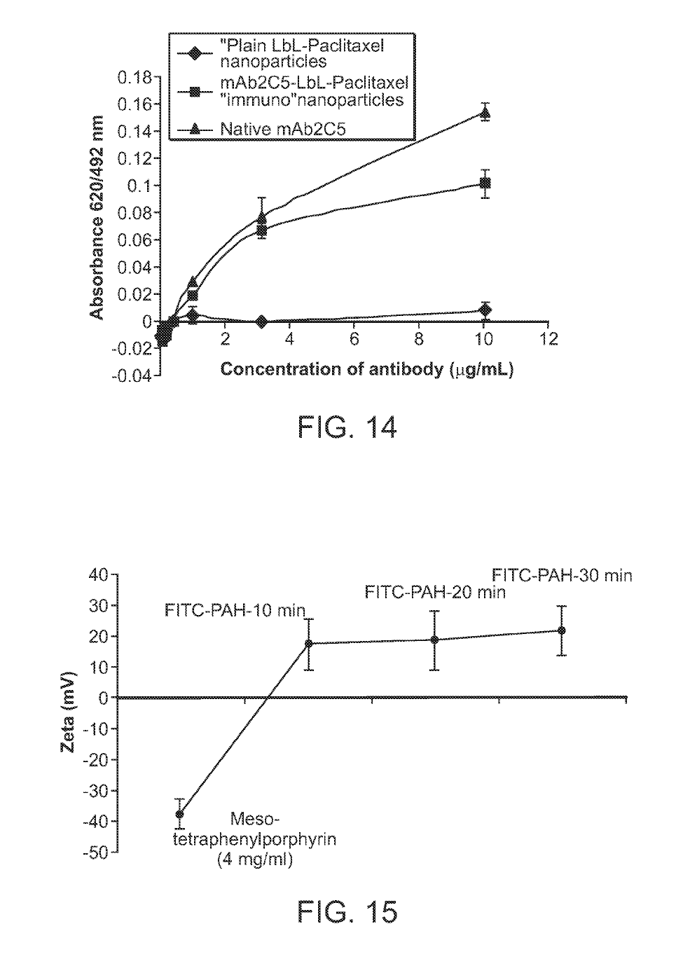

To demonstrate the ability to derivatize the LbL-coated drug nanoparticles, paclitaxel-containing nanoparticles were produced having one layer of PAH, as described above. The tumor-specific mAb 2C5 was then attached to the PAH-coated paclitaxel nanoparticles via free amino groups on the surface layer of PAH. As depicted in FIG. 14, 2C5-modified LbL-coated paclitaxel nanoparticles specifically recognized the target antigen (i.e., nucleosomes).

The cytotoxicity of the mAb 2C5-modified paclitaxel-containing nanoparticles was determined using MCF-7 cells and BT-20 cells, as described above. Paclitaxel nanoparticles having a single layer of PAH, but without the 2C5 modification, were used as control. After incubating MCF-7 cells for 48 hrs or 72 hrs in the presence of 100 ng/mL unmodified paclitaxel nanoparticles, about 95% of the cells were alive. However, when MCF-7 cells were incubated in the presence of 100 ng/ml 2C5-modified paclitaxel-containing nanoparticles, around 30% of the cells were killed. Similar results were seen when BT-20 cells were incubated in the presence of 30 ng/ml of paclitaxel nanoparticles.

### Example 2

### Preparation of Stable Nanoparticles of meso-Tetraphenylporphyrin and Camptothecin

LbL nanoparticles of meso-tetraphenylporphyrin and camptothecin were prepared as described in Example 1. As depicted in FIG. 15, meso-tetraphenylporphyrin nanoparticles were produced using a coating of FITC-labeled PAH, which reversed the surface charge from negative to positive. SEM demonstrated particle sizes from about 83 nm to about 194 nm (FIG. 15B).

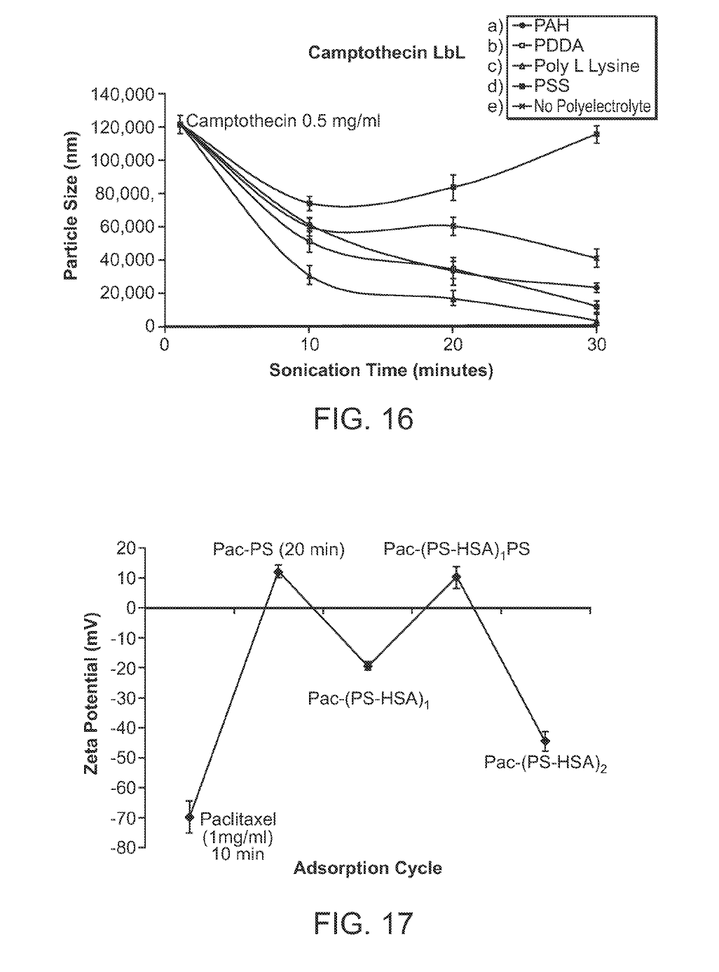

LbL nanoparticles of camptothecin were also prepared. Optimization of the first polycation coating was performed. Three polycations (PAH, PEI and PDDA) and one polyanion (PSS) were used. In presence of PSS, which has the same charge as the drug core, no particle size decrease was observed (FIG. 16). All the polycations were able to reduce the particle size, and the smallest particles were obtained with polylysine treatment. SEM images of camptothecin after 30 mins of sonication with cationic poly L-lysine detected particles of about 390 nm, whereas sonication with PSS resulted in larger particles.

Some representative results are summarized below in Table 1. The release time for tamoxifen was about 6 hours.

TABLE 1

| | Particle | Coating | |
|---|---|---|---|
| Drugs | Size | Thickness | Colloidal Stability |
| Tamoxifen | 125 ± 30 nm | 5 nm | at least one week |
| Paclitaxel | 110 ± 30 nm | 5 nm | at least one week |
| meso-Tetraphenylporphine | 140 ± 50 nm | 5 nm | at least one week |
| Camptothecin | 390 ± 50 nm | 5 nm | at least one week |

### Example 3

### Preparation of Stable Nanoparticles of Paclitaxel Using Biocompatible Coatings

LbL drug nanoparticles of paclitaxel were prepared as described in Example 1, but biocompatible materials were used in the coatings. Paclitaxel-containing nanoparticles were prepared with a first layer of protamine sulfate (PS) followed by subsequent coatings of human serum albumin (HSA). Smaller nanoparticles were obtained with 30 min sonication +LbL coating with protamine sulfate.

FIG. 17 depicts zeta potential readings of paclitaxel LbL by biocompatible PS and HSA. As demonstrated, the charge alternates between positive and negative values with each subsequent addition of PS and HSA, respectively.

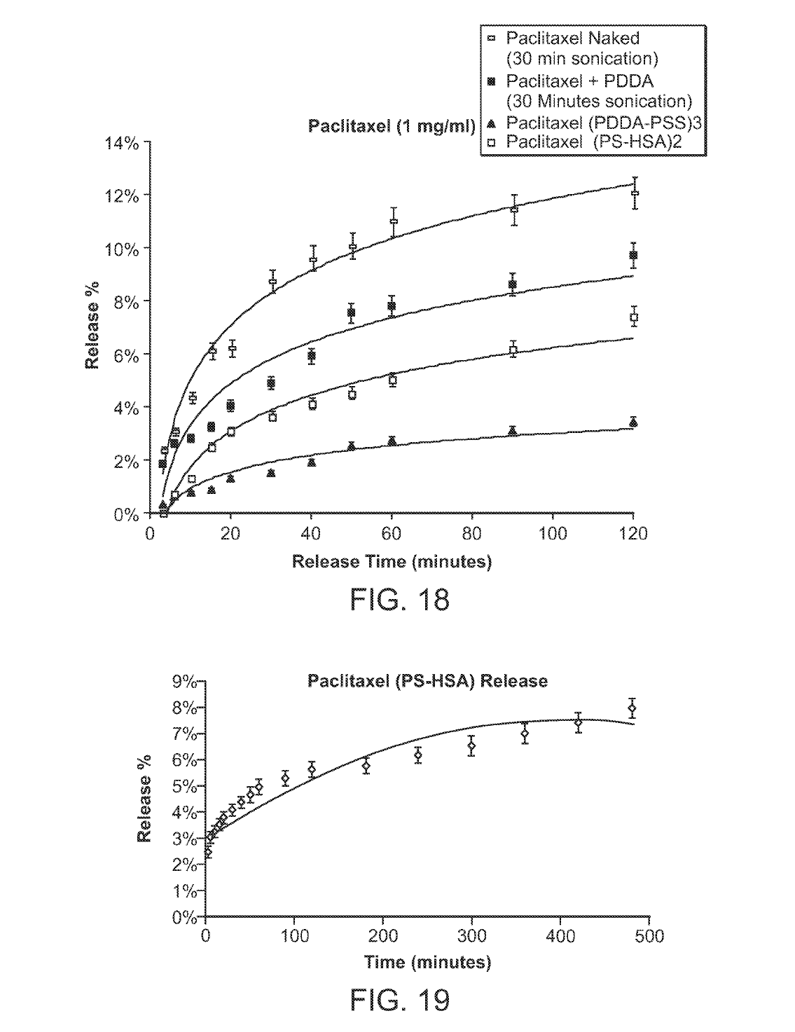

To determine the release of paclitaxel from these nanoparticles, the release rates through 200 nm membranes over 2 hrs were measured, as described in Example 1. As shown in FIG. 18, at 2 hrs, 12.06% paclitaxel was release from naked paclitaxel with sonication. 9.7% of paclitaxel was released from particles with 1 layer of PDDA, 7.41% paclitaxel was released from particles having two (PS-HSA) bilayers, and 3.44% paclitaxel was released from particles having three (PDDA-PSS) bilayers.

FIG. 19 depicts the sustained release curve for paclitaxel coated with 3 bilayers of biocompatible PS and HSA for 8 hrs at sink conditions at pH 7.3. As demonstrated, these nanoparticles have sustained release for over 500 mins.

### EQUIVALENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A stable nanoparticle comprising:
(a) a compound;
(b) a first defined solid polymeric layer comprising a first polymer, the first layer
surrounding the compound; and
(c) a second defined solid polymeric layer comprising a second polymer, the second layer surrounding the first layer,
the first polymer and the second polymer having opposite charges, and the nanoparticle having a diameter of about 100 nm to about 500 nm.

2. The nanoparticle of claim 1, wherein the compound is present at about 5% by weight to about 95% by weight.

3. The nanoparticle of claim 1, wherein the first polymeric layer and the second polymeric layer have a combined thickness of about 5 nm to about 30 nm.

4. The nanoparticle of claim 1, wherein the first polymer is positively charged and the second polymer is negatively charged.

5. The nanoparticle of claim 1, wherein the first polymer is negatively charged and the second polymer is positively charged.

6. The nanoparticle of claim 1 comprising more than two defined, solid, polymeric layers.

7. The nanoparticle of claim 1, further comprising a third polymeric layer surrounding the second polymeric layer, the third polymeric layer comprising a third polymer having an opposite charge from the second polymer.

8. The nanoparticle of claim 7, wherein the first polymer and the third polymer are the same.

9. The nanoparticle of claim 7, further comprising a fourth polymeric layer surrounding the third polymeric layer, the fourth polymeric layer comprising a polymer having an opposite charge from the third polymer.

10. The nanoparticle of claim 1, wherein the second polymeric layer is modified with a targeting agent.

11. The nanoparticle of claim 7, wherein the third polymeric layer is modified with a targeting agent.

12. The nanoparticle of claim 9, wherein the fourth polymeric layer is modified with a targeting agent.

13. The nanoparticle of claim 10, wherein the targeting agent is an antibody.

14. The nanoparticle of claim 1, wherein the nanoparticle does not contain a detergent or a surfactant.

15. The nanoparticle of claim 1, wherein the compound is released from the nanoparticle at a rate of 7% within about two hours.

16. The nanoparticle of claim 12, wherein the compound is released from the nanoparticle at a rate of about 3% with about two hours.

17. A nanoparticle comprising:
(a) a compound; and
(b) a polymeric coating comprising alternating polymeric layers of oppositely charged polymers,
the nanoparticle having a diameter of about 100 nm to about 500 nm.

18. The nanoparticle of claim 17, wherein the nanoparticle comprises two or more layers of oppositely charged polymers.

19. The nanoparticle of claim 17, wherein the compound is present at about 5% by weight to about 95% by weight.

20. The nanoparticle of claim 17, wherein the polymeric layers have a combined thickness of about 5 nm to about 30 nm.

21. A method of making a stable nanoparticle, the method comprising:
subjecting a water-insoluble compound to ultrasonication; and
adding a first polymer to the compound in the presence of ultrasonication,
the polymer added at a concentration sufficient to form a stable first polymeric layer around the compound.

22. The method of claim 21, wherein after ultrasonication, the water-insoluble compound has a negative charge in the absence of the polymer.

23. The method of claim 21, wherein the polymer added to the compound has a positive charge.

24. The method of claim 21, wherein the ultrasonication is performed at about 20° C. to about 30° C.

25. A method of treating a subject having a tumor, the method comprising administering to the subject a nanoparticle in an amount sufficient to reduce tumor size or number of tumor cells, wherein the nanoparticle comprises:
(a) a compound;
(b) a first defined solid polymeric layer comprising a first polymer, the first layer surrounding the compound; and
(c) a second defined solid polymeric layer comprising a second polymer, the second layer surrounding the first layer,
the first polymer and the second polymer having opposite charges, and the nanoparticle having a diameter of about 100 nm to about 500 nm.
